(19) United States
(12) Patent Application Publication
(10) Pub. No.: US 2017/0135845 A1
(43) Pub. Date: May 18, 2017

(54) Elastic Pressure Device

(71) Applicant: Martin Alan Cohen, Rosarito BC (MX)

(72) Inventor: Martin Alan Cohen, Rosarito BC (MX)

(21) Appl. No.: 14/859,333

(22) Filed: Sep. 20, 2015

(51) Int. Cl. A61F 5/41

(52) U.S. Cl. CPC A61F 5/41; A61F 2005/411; A61F 2005/414

(57) ABSTRACT

A flexible, elastic, theraputic, pressure device for men. Two flexible bars are affixed to a flexible soft containment apparatus using superficial pressure meaning toward or at the body surface laterally, along two sides of the penile shaft longitudinally. The device does not reach as far as the glans corona at its distal edge.

### FIELD OF THE INVENTION

A useful and new theraputic device for men that improves a cognitive state by a redirection of affective and effective nerve impulses, being an apparatus for the genitals enhanced with reflex point protrusions to effect a change in consciousness.

### PRIOR ART

Prior art specifically relating to the present invention seems scarce Muscle and tendon support devices are in existence that use straps that wrap around a body part and use support elastic straps have received patents such as U.S. Pat. No. 8,608,677 for use on arm, leg or hip, called Device For Carrying the Load of Injured Soft Tissue. A commercial knee device trademark name BeActive wraps around the upper calf and knee area and incorporates a pressure pad to activate a reflex point or accupressure point to lessen sciatic back pain. It is described by its designers as a pressure leg brace U.S. Pat. No. 8,187,212. The use of a pressure pad agrees in form with the current invention if not in function as it conceives of an activating point not obvious to a related effect. In function there seem no related consiousness altering devices by this method as with the present invention. Patents exist for mechanical devices covering the penis that serve to gradually stretch and lengthen it. A vacuum design has a similar purpose. Condoms of course are a stretchable penile covering for preventing the escape of semen. Aritficial prepus forms have been designed to give the sensation of being uncircumcized to men. None of these use pressure at specified points or aim at a cognitive effect as described. The proper nitch for the present invention has remained elusive. Subclass A61M allows for devices by means of a particular sensory stimulus to cause a change of consciousness below the threshold of consciousness. The Elastic Pressure Device for men does enact a change below the threshold of awareness, but it results in a shift in the conscious state. The rods or bars being larger exert kinetic energy upon the body while the sleeve or containment being equal in diameter to the body part has only potential energy. The bars do function as an energy distributor in this interpretation. The subclass mentioned relates to introducing what is called ‘media’ onto the body. This term has a fairly wide range of meanings. It could mean a lotion, a vibratory pad, lazer light, sound waves and perhaps even pressure bars affixed to a body part. Subclass A61H includes apparatus or processes that ‘restore to health or alleviate the effects of sickness or other types of abnormal conditions or bodies or their parts’ that is devices, that use mechanical energy directly applied to human bodies by physical contact for improving their functioning that ‘are not specifically provided for elsewhere in another subclass’. The present invention may fit into the A61H subclass. A clarification is needed as to the present invention being a physical therapy apparatus as mentioned under this subclass ; ‘devices for locating or stimulating reflex points in the body’. A61H 19/34 is described as ‘apparatus for the genitals with enhanced rubbing effect’, apparently referring to sexual stimulation rather than a neurological effect.

### DESCRIPTION

### Background

Two states of cognitive perceptual awareness result from neurological triggers. One is inherent in the individual a priora while the second is triggered via intromission via a redirection of reentrant neuronal group selection. The inherent state is masturbatory. The second triggered state is not accessable other than intergenital contact also known as intromission between a man and a woman where a male prophylactic is not employed. This second state called intercourse-mode, intromission-mode or IN-mode is often a condition of greater energy and decreased depression for both men and women.

Men who are aged, widowed, non-heterosexual, single with moral objections to non-marital sex, disabled, suffering from injury, between relationships or for any reason without a female partner are thus unable to achieve the intermissive state of being. Any male who does can only remain so until masturbation or nocturnal emission return them to their inherent state. This is equally the case in circumsized and non-circumsized men. This occurrence has been known as sexual-mode theory also as sex-modes. Two different sexual modes exist in both men and women. One is called masturbation-mode and the other is intercourse-mode. Nature provides this to make men more suited to family life as an evolutionary process that is purposive. As technology has replaced nature we have lost much of our connection with nature. In nature, once a creature procreates its function is more or less over and it can then die. Humans however have developed in a way that this is far from the case. Ones usefulness in society is no longer defined by parental status. Relationships have grown to include the LGBT community as well as marriage of same gender couples. Additionally divorce, illness and personal tragedy can leave the male alone without a way of accessing IN-mode.

### SUMMARY OF THE INVENTION

Two pressure areas on the sides of the male penis are able to mimic IN-mode even though a man is still in masturbation-mode. It does not function as a ‘trigger’ as with the intermissive state of being that will. switch a man so the pressure can then be ended leaving him in IN-mode. The device is in a form comfortable enough for daily wear that maintains key activating areas. A man already in IN-mode utilizing this device will not experience this effect, but if his last sexual release was not conventional heterosexual intermissive sex then he is in MB-mode or masturbation-mode and wearing it should be noticeable as enhanced clarity of mind, positive body sensations and emotions that much more imitate IN-mode than MB-mode. By the use of a fairly simple device, neurocircuts specific to the intermissive state are accessed as soon as the device is utilized. This state ceases whenever the device is disengaged.

### IN THE DRAWINGS

FIG. 1 is a perspective drawing of the invention, rendered as to illustrate the various components and their arrangement viewed from below;

FIG. 2 is to show the components, separate and unassembled, for illustration purposes;

FIG. 3 is a cross-section of the proximal end of the sleeve as well as the other components and how they are connected according to the invention;

FIG. 4 shows a theraputic pressure device in perspective as would be worn to reference it with the human body;

FIG. 5 is a partial drawing giving a clear view of the strap-loops and their situation according to the invention.

FIG. 6 is a cross-section of only the rods and sleeve to explain the pressure increasing nature of the rods.

FIG. 7 is a one-piece unit of soft synthetic materials that shows a different embodiment yet consistent with the functional elements of the invention.

FIG. 8 shows an embodiment incorporating bars to a sleeve with pressure directed inward from outside the sleeve made possible by small sleeves and the stretchablility of the fabric.

### DETAILED DESCRIPTIONT

A flexible elastic theraputic pressure device consisting of five parts.

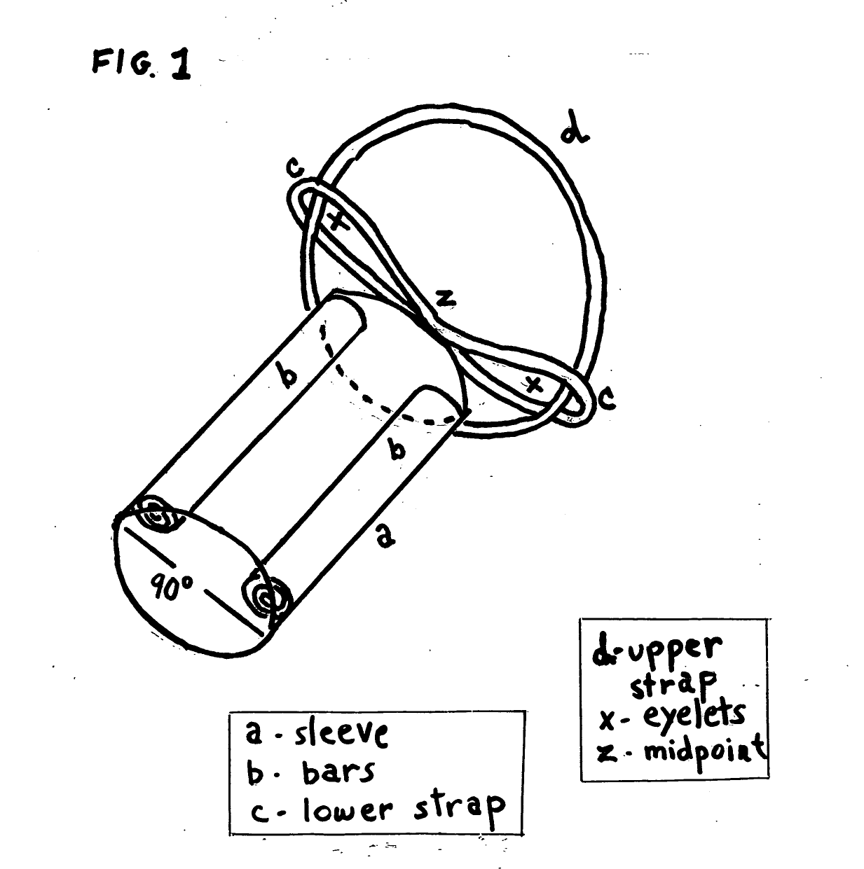

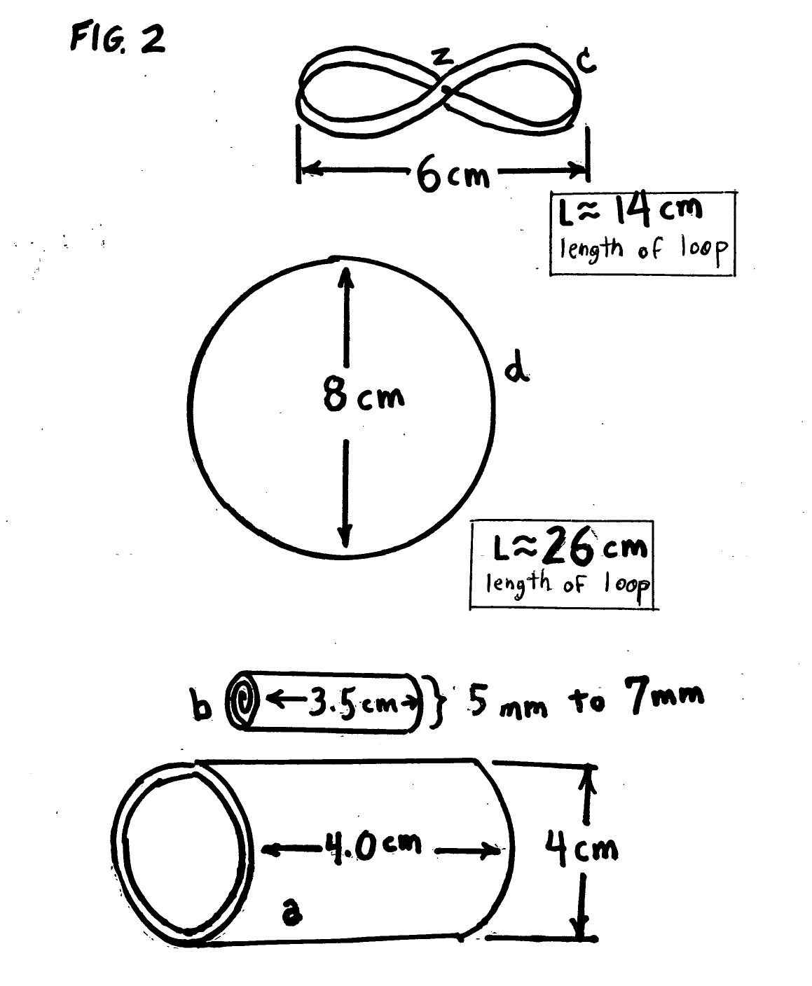

This embodiment includes a single flat rectangular sheet of elastic, breathable, bandage-type, high compression fabric. This is sewn together at its shorter ends to form a sleeve that serves as a containment apparatus for the rods [a] FIGS. 1 and 2, that is circumferiantally stretchable, that covers the penile shaft excepting

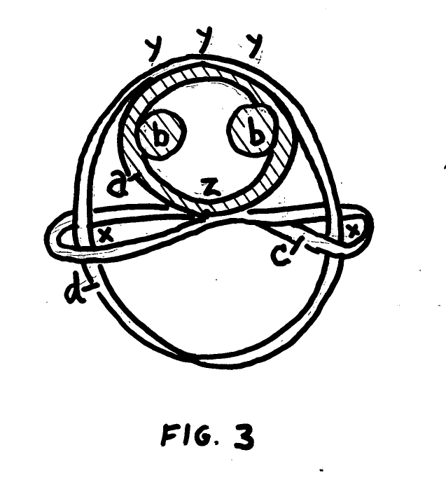

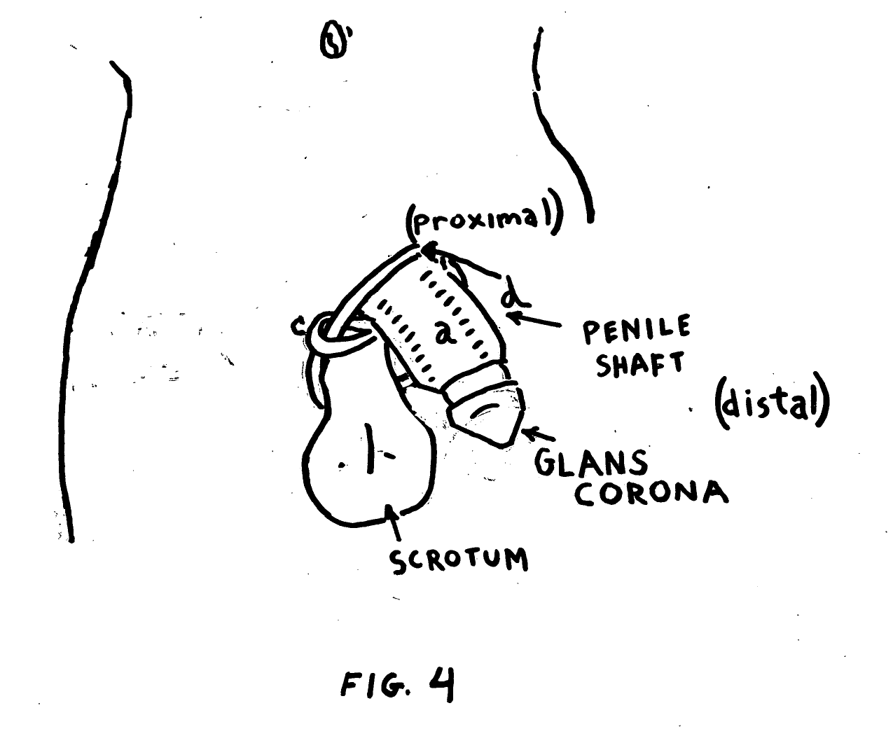

the glans FIG. 4, and contains two rolled, sewn, lengths of similar fabric to form flexible rods or bars [b] FIG. 2, fixed longitudinally on opposite sides of the sleeve on its inner surface. FIGS. 1 and 3 one centemeter [1 cm] shorter than the sleeve length, thus slightly recessed from the sleeve ends FIG. 2.

Two stretchable anchoring straps [c] and [d] osculate the proximal end of the sleeve's exterior FIGS. 1 and 3. The first strap is attached to the underside of the sleeve in the shape of a loop, sewn at a midpoint area [z] between the two bars FIGS. 1 and 3.

The second or longer strap-loop is sewn to the sleeve circumferentially at the proximal end of the upper half of the sleeve [y] FIG. 3 extending downward around the sleeve and through the two eyelets [x] of the lower securing strap that have been formed by its attachment at point labled ‘z’ shown at two opposite points on the lower loop to the sleeve in the FIGS. 1 and 3.

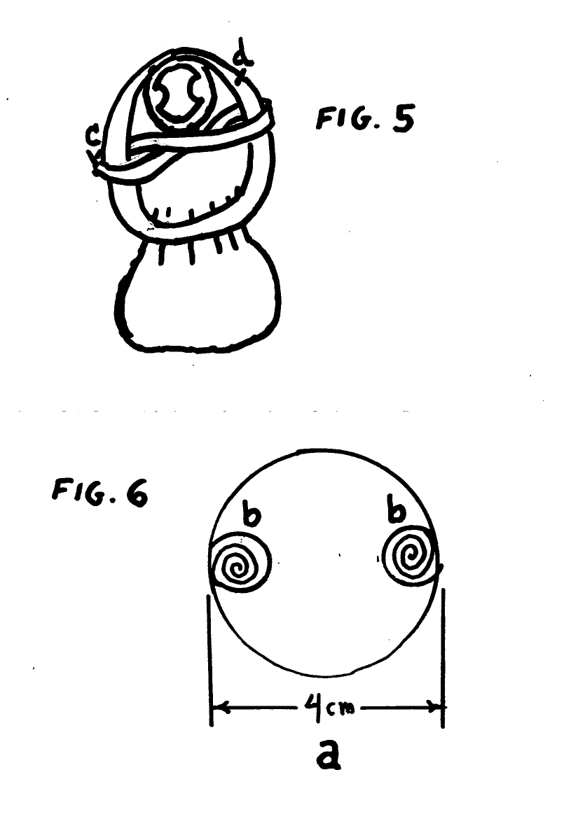

The upper securing strap is then anchored around and under the scrotal sac to help prevent shifting of the sleeve up the penile shaft FIG. 4 and FIG. 5 in which the device is viewed as if from behind.

All the components can be conjoined by stitching although other bonding methods such as adheasives could be employed. The fabric can be of a material such as high compression elastic bandage thin enough to be comfortable. This is intended as an external device producing a unique linear pressure. It covers part of the male genitilia held in place by elastic straps. This device is intended for daily wear to effect an alteration of consciousness and cognitive function for males who might be aged, widowed, disabled, with communicable or degenerative diseases or those wishing a more active and energetic life pertaining to the intromissive state of being.

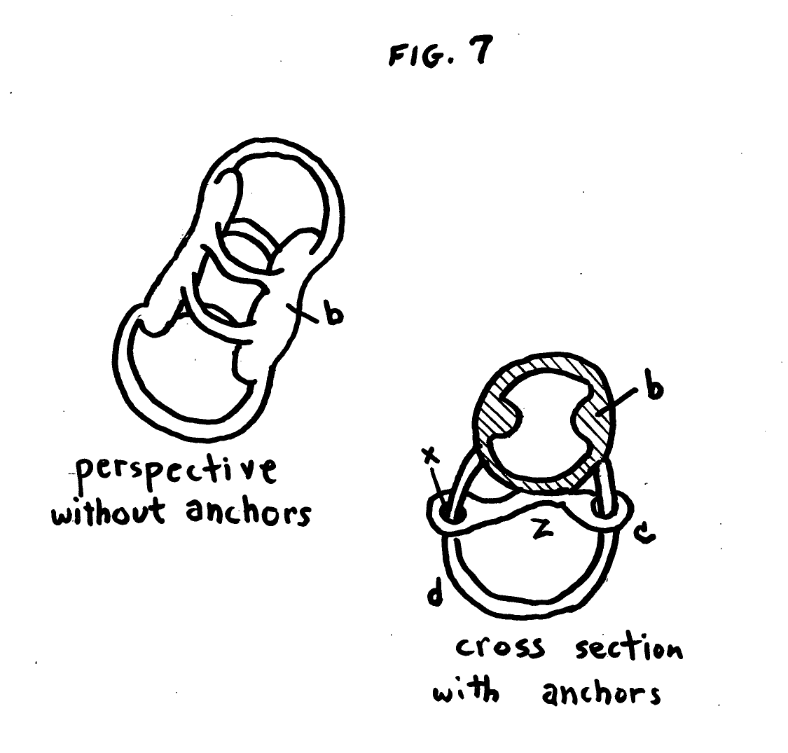

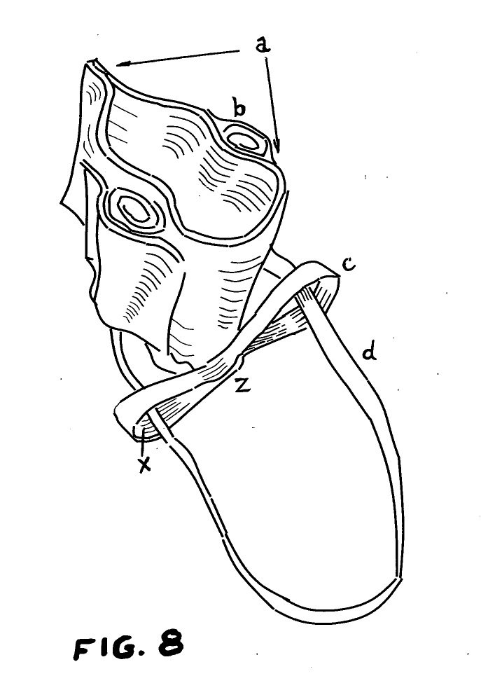

Although it is referred to as an elastic pressure device it in no way functions as an injury, tissue or joint support device, nor does it attempt to permanently correct imbalances but to mimic an effect attainable via intromission known as IN-mode. achieved by a constant pressure from two flexible bars parallel to one another intervolved within an elastic fabric sleeve-and-anchor interlink covering the penile shaft, save for the glans corona at its distal end. Two bars located on the interior or exterior surface diminish the space within the diameter of the sleeve, having a diameter of 4 cm. Each bar being about 1 cm in diameter in an uncompressed state will compress to 5 mm to 7 mm depending on the size used FIG. 6, designed to increase longitudinal pressure within the sleeve. Staistical average data for the average penis is a mean of 3.9 cm diameter and 9.3 cm in circumference. The effects upon the nervous system alter cognitive perception. The soft materials are intended to render this device not to be felt or sensed by the wearer as a pair of contact lenses might while worn, with the compressibility and variability of the bars held in place by a containment structure allowing for variability. The overall device can easily be produced in a variety of sizes. As the present invention is embodied in different forms the illustrations are not to limit its form only as construded to illustrate. Other forms are also possible that function equally within the parameters of essential characteristics and the scope of the invention within the stated claims. The bars can be made of the same stretchable fabric as the sleeve that are then rolled and sewn or glued to form the bars. Bars may be sewn to the inside of the sleeve. In another form the bars are slipped into small pockets made of squares of the stretch material 3 cm by 3 cm locatable on the outside of the sleeve and the lessening of diameter at the bar areas is pushed inward into the sleeve. This allows the wearer to replace bars made slightly larger around substituted for thinner bars. In this way a permutation of three sizes is possible using in one case a thicker bar made from 4 cm then rolled bar and one thinner bar, two thinner bars or two thicker bars of 6 cm fabric then rolled into bars that are glued or sewn to retain their shape. This allows a wearer to make adjustment to allow for individual preferences as to comfort and affect. A flexible anchoring strap may be of synthetic or woven material, sewn or glued and so forth, therefore include but are not limited to these methods alone. Even two horse shoe rings with the two pressure bars thus held in place will cause the theraputic cognitive effect if made to be comfortable and able to remain in place acceptably well.

We claim:

1. An elastic pressure device for men by means of two flexible bars equidistant from an imaginary longitudinal axis at the center of a containment apparatus which by their diameter and length effect the neurocircuts in positive ways mimicing what is called the intromissive state of being.

2. A method of cognitive effect being caused by these two bars where little pressure results from the resistance against the penile shaft by the elastic sleeve where the two bars exert a slightly greater pressure along both sides of the penile shaft relative to the sleeve; the bars being attached to the containment apparatus where the said effect being cognitve results from relative differences in pressure of the bars.

3. An elastic pressure device wherein two straps affixed at the proximal end of a sleeve adjacent an area where the penile shaft connects to the body anchoring the sleeve from riding up the base of the penile shaft where the upper strap is a stretchable loop and is attached along the upper half of the outside of the sleeve so one strap is stretched so it reaches over then contracts comfortably around the scrotal sac while the lower strap is a smaller loop attached at an area on the underside proximal end of the sleeve at its midpoint area forming two eyelets through which the larger or longer loop is passed on either side.
